(19) United States
(12) Patent Application Publication
(10) Pub. No.: US 2026/0000153 A1
(43) Pub. Date: Jan. 1, 2026

(54) DIGITALIZED THERAPEUTIC FOOTWEAR WITH ADAPTIVE SAMPLING, EDGE AI, FEDERATED LEARNING AND ENERGY HARVESTING

(71) Applicant: KARAN JOGESH BHASIN, MUMBAI (IN)

(72) Inventor: KARAN JOGESH BHASIN, MUMBAI (IN)

(21) Appl. No.: 19/318,480

(22) Filed: Sep. 4, 2025

Related U.S. Application Data
(63) Continuation-in-part of application No. 18/036,668, filed on May 12, 2023.

(30) Foreign Application Priority Data
Mar. 7, 2022 (IN) 202221012073

(51) Int. Cl. A43B 3/44; A43B 3/40; A61B 5/00; A61B 5/0205; A61B 5/103; G16H 40/67; H04W 12/02

(52) U.S. Cl. CPC A43B 3/44; A43B 3/40; A61B 5/02055; A61B 5/1038; A61B 5/486; A61B 5/6807; A61B 5/7267; A61B 5/7275; A61B 5/7455; G16H 40/67; H04W 12/02; A61B 2560/0214

(57) ABSTRACT

The invention discloses a therapeutic footwear system comprising a footwear body (101) with insole (102), midsole (103), and outsole (105), integrated with a multi-modal sensor array (110) including a plantar pressure matrix (111), inertial measurement unit (112), photoplethysmography sensor (113) and temperature sensor (114). An embedded processor (120) houses an adaptive sampling module (121), an edge-AI inference engine (122), a federated learning client (123), and memory (124) for local analysis. A feedback interface (130) with zonal haptic actuators (131) under medial forefoot (132), lateral forefoot (133), medial heel, and lateral heel (135) delivers corrective prompts. A power subsystem (140) comprising a battery management circuit (141), energy harvesting elements (142), and battery (143) sustains operation. The integration reduces latency, conserves energy, preserves privacy, and enables closed-loop therapeutic intervention during ambulation.

### CROSS REFERENCE

The present invention is an improvement of a co-pending patent application in India with application No. 202221012073 filed on Jul. 3, 2022 with title “Digitalized Therapeutic Footwear”.

### TECHNICAL FIELD OF THE INVENTION

The present invention relates to the field of wearable biomedical and therapeutic systems, and more particularly to digitalized therapeutic footwear incorporating multi-modal physiological and biomechanical sensors, embedded processors with adaptive sampling and edge-based artificial intelligence, federated learning for privacy-preserving model updates, zonal haptic feedback for corrective intervention, and hybrid energy harvesting subsystems for sustained operation.

### BACKGROUND OF THE INVENTION

Therapeutic footwear has traditionally been designed to provide structural support, cushioning, and pressure offloading for patients suffering from orthopaedic, neurological, or metabolic disorders, such as diabetic neuropathy, plantar fasciitis, or post-surgical rehabilitation requirements. While these conventional solutions address static biomechanical needs, they are inadequate for dynamically monitoring the evolving physiological and biomechanical conditions of the wearer. In parallel, the advent of wearable technologies has introduced smart shoes and wrist-based devices that embed sensors for activity tracking, gait analysis, and basic health monitoring. However, these systems are limited in scope, focusing primarily on simple parameters such as step counts, walking speed, or gross activity levels, without addressing therapeutic feedback or personalized corrective interventions.

Existing prior art, such as WO2023170536A1, discloses digitalized therapeutic footwear incorporating multiple sensors embedded in the insole or midsole to capture physiological and biomechanical data. While this earlier development introduced the important concept of combining plantar pressure sensors, inertial sensors, and physiological measurement modules in a footwear platform, its functionality remains largely dependent on raw data collection and transmission. Similar prior art references, including US20220000393A1 and other known smart footwear systems, rely on centralized or cloud-based processing architectures to analyse data, which introduces latency, dependency on network connectivity, and serious concerns regarding data privacy. Moreover, these systems operate on fixed-rate sensor sampling regimes that continuously poll sensors irrespective of user condition, leading to inefficient energy usage and reduced battery runtime.

The disadvantages of these prior systems are significant in therapeutic applications. First, reliance on cloud processing leads to latency, which prevents real-time intervention critical for fall prevention or ulcer risk management. Second, fixed-rate sampling results in excessive energy consumption, requiring frequent recharging and limiting long-term usability. Third, centralized machine learning approaches demand transmission of raw personal health data to remote servers, raising serious privacy and regulatory concerns under frameworks such as GDPR and HIPAA. Fourth, the absence of closed-loop correction limits their therapeutic utility; most prior systems act as passive monitors rather than delivering actionable feedback to the wearer. Finally, none of the prior solutions integrate energy harvesting subsystems into footwear to extend operational life, resulting in user fatigue, compliance issues, and limited adoption in clinical practice.

Accordingly, there exists a dire and unmet need for an improved therapeutic footwear system that not only collects physiological and biomechanical signals but also performs adaptive, real-time processing directly on the device, thereby eliminating dependency on cloud infrastructure. Such a system must incorporate adaptive sampling mechanisms to minimize unnecessary energy expenditure, while still capturing high-resolution data during critical events. Further, it must support federated learning approaches to enable distributed intelligence across multiple devices, allowing the models to continuously improve without ever compromising personal health data privacy. Additionally, the system must provide closed-loop corrective feedback, for example through haptic actuators embedded under specific plantar zones, to enable the wearer to redistribute load, correct gait abnormalities, or adhere to rehabilitation protocols in real time. To ensure long-term operation without frequent charging, the system should also integrate energy harvesting technologies, such as piezoelectric or thermoelectric transducers, to supplement battery supply during ambulation.

Thus, the present invention seeks to overcome the deficiencies of the prior art by providing a digitalized therapeutic footwear system that is energy-efficient, privacy-preserving, and capable of real-time, closed-loop intervention. By integrating adaptive sampling, edge-based artificial intelligence inference, federated learning, zonal haptic feedback, and hybrid energy harvesting into a single wearable platform, the invention addresses long-standing challenges in diabetic monitoring, elderly fall prevention, athletic optimization, and post-surgical rehabilitation. This combination of technical features represents a synergistic advancement over the state of the art, delivering an intelligent therapeutic solution that is both clinically impactful and user-friendly.

### OBJECTS OF THE INVENTION

It is an object of the present invention to provide an improved therapeutic footwear system that integrates multi-modal physiological and biomechanical sensing with advanced embedded processing, so that the condition of the wearer's foot health, gait, and overall lower-limb biomechanics can be monitored with high fidelity during ambulation and rest. By embedding sensors such as plantar pressure matrices, inertial measurement units, photoplethysmography sensors, and temperature probes directly within the insole and midsole, the invention aims to capture a rich set of signals required for accurate therapeutic assessment.

Another object of the invention is to overcome the limitations of fixed-rate sampling found in prior art devices by introducing an adaptive sampling module built into the embedded processor. This adaptive sampling mechanism allows the system to dynamically modify sensor duty cycles in response to user-specific deviations such as gait asymmetry, abnormal plantar temperature differences, or irregular cadence. By adjusting sampling only when needed, the invention seeks to conserve energy while still providing detailed measurements at critical moments.

A further object of the invention is to provide real-time, low-latency analysis of the sensed signals by incorporating an edge-based artificial intelligence inference engine within the footwear itself. Unlike conventional systems that depend on cloud computing, the present invention is designed to process data locally on an embedded microcontroller or system-on-chip with neural compute capability. This ensures that corrective actions can be generated immediately, even in the absence of network connectivity, thereby delivering therapeutic interventions without delay.

Another important object of the invention is to enhance collective learning and personalization across multiple footwear devices through the implementation of a federated learning client. This client enables each device to train its local model on user-specific data while transmitting only anonymized parameter updates to an aggregation server. By doing so, the system achieves the dual goals of continuously improving predictive accuracy across a population while preserving the privacy of individual users, as raw physiological data never leaves the footwear.

It is also an object of the invention to provide closed-loop therapeutic feedback to the wearer in the form of localized haptic prompts. By embedding multiple haptic actuators under different plantar zones, including medial forefoot, lateral forefoot, medial heel, and lateral heel, the invention seeks to deliver region-specific corrective cues that guide the user to redistribute load, adjust stride length, or alter cadence in real time. Such corrective prompts improve patient compliance in rehabilitation, reduce risk of diabetic foot ulcers, and assist in fall prevention among elderly users.

Another object of the invention is to address the problem of limited battery life in wearable devices by integrating an energy harvesting subsystem into the outsole of the footwear. By employing piezoelectric, electromagnetic, or thermoelectric transducers that convert mechanical and thermal energy generated during walking into electrical power, the invention aims to supplement the onboard battery and extend the operational life of the system to at least twenty-four hours in adaptive sampling mode.

A further object of the invention is to ensure that the combination of adaptive sampling, edge-AI inference, federated learning, closed-loop haptic guidance, and energy harvesting produces a synergistic effect that goes beyond the sum of its parts. By reducing latency, conserving energy, and preserving user privacy simultaneously, the invention provides a practical, clinically useful, and user-friendly therapeutic footwear platform.

In this manner, the present invention seeks to deliver a comprehensive digitalized therapeutic footwear system that directly addresses the shortcomings of prior art, while enabling new levels of monitoring, feedback, and personalization in the management of diabetic care, fall prevention, athletic training, and post-surgical rehabilitation.

### BRIEF SUMMARY OF THE INVENTION

In one aspect of the present invention, there is provided a therapeutic footwear system that integrates a plurality of physiological and biomechanical sensors within the insole and midsole to continuously acquire signals from the plantar surface and the lower limb of the wearer. The multi-modal sensor array may include a plantar pressure matrix for determining load distribution and center-of-pressure trajectories, an inertial measurement unit for gait and motion analysis, a photoplethysmography sensor for capturing cardiovascular signals such as heart rate variability, and one or more temperature sensors for detecting plantar thermal asymmetry associated with inflammatory conditions.

In another aspect of the invention, the therapeutic footwear incorporates an embedded processor housed within the footwear body, the processor being configured with an adaptive sampling module. The adaptive sampling module operates to dynamically regulate sensor duty cycles, thereby increasing or decreasing polling rates in response to deviations from stored baseline parameters. This permits the system to conserve energy during normal conditions while capturing high-resolution data during events such as gait instability, prolonged stance phase, or abnormal temperature gradients.

In a further aspect of the invention, the embedded processor is provided with an edge-based artificial intelligence inference engine that executes predictive analytics locally within the footwear. Unlike prior systems that rely on remote servers or cloud-based computation, the present invention ensures low-latency, on-device decision-making.

This allows the footwear to generate therapeutic outputs instantaneously, even when network connectivity is unavailable, thereby ensuring uninterrupted monitoring and intervention.

In yet another aspect of the invention, the therapeutic footwear system includes a federated learning client embedded within the hardware memory of the processor. This client allows each footwear device to train a local model on its user-specific data and to transmit only parameter updates to an external aggregation server. In this way, the system enables population-level model improvements while retaining raw physiological data locally on the footwear, thus preserving privacy and complying with regulatory requirements for sensitive medical information.

In an additional aspect, the invention provides a feedback interface comprising multiple haptic actuators embedded under predefined plantar zones of the insole. These actuators are capable of delivering localized corrective prompts that correspond to the type and location of the detected anomaly. By applying discrete haptic signals to the medial forefoot, lateral forefoot, medial heel, or lateral heel, the system guides the wearer to redistribute load, adjust stride length, or alter cadence in real time, thereby delivering closed-loop therapeutic intervention.

In another aspect, the footwear system incorporates a power management subsystem coupled with energy harvesting elements embedded in the outsole. These elements may include piezoelectric, electromagnetic, or thermoelectric transducers configured to convert mechanical or thermal energy generated during walking into electrical energy. The harvested energy supplements the rechargeable battery, thereby extending the operational lifetime of the system and supporting continuous functionality without frequent recharging.

In yet another aspect, the invention encompasses a method of providing therapeutic monitoring and corrective intervention using the footwear system. The method includes acquiring multi-modal sensor data, comparing it with stored baseline values, adaptively modifying sampling rates when deviations are detected, performing real-time inference on the embedded processor, actuating haptic feedback to deliver corrective cues, and transmitting anonymized parameter updates for federated learning.

Taken together, these aspects of the invention establish a digitalized therapeutic footwear system that achieves a synergistic technical effect by combining adaptive sampling, edge-based inference, federated learning, zonal haptic feedback, and energy harvesting in a single wearable platform. This integration reduces latency, conserves energy, preserves user privacy, and provides actionable therapeutic guidance, thereby overcoming the shortcomings of prior art and addressing critical needs in diabetic management, elderly fall prevention, sports performance optimization, and post-surgical rehabilitation.

### BRIEF SUMMARY OF THE DRAWINGS

The invention will be further understood from the following detailed description of a preferred embodiment taken in conjunction with an appended drawing, in which:

FIG. 1 illustrates a schematic block diagram of the therapeutic footwear system, showing the footwear body, insole, midsole, outsole, multi-modal sensor array, embedded processor with adaptive sampling module, edge-AI inference engine, federated learning client with memory, feedback interface with haptic actuators, and the power subsystem comprising battery management circuit and energy harvesting elements.

FIG. 2 shows a cross-sectional view of the footwear body, indicating placement of the plantar pressure matrix, inertial measurement unit, photoplethysmography sensor, and temperature sensor within the insole and midsole. The figure also shows the arrangement of zonal haptic actuators under the medial forefoot, lateral forefoot, medial heel, and lateral heel regions.

FIG. 3 depicts the operational flow of the system, including acquisition of signals from the multi-modal sensor array, adaptive modification of sampling by the module, processing by the embedded processor with edge-AI inference engine, generation of corrective outputs, actuation of haptic feedback actuators, and transmission of parameter updates via the federated learning client.

FIG. 4 illustrates an exemplary embodiment of the power subsystem integrated into the outsole, showing energy harvesting elements such as piezoelectric and thermoelectric transducers, electrically coupled to the battery management circuit for supplementing the rechargeable battery during ambulation.

### DETAILED DESCRIPTION OF THE INVENTION

In an exemplary embodiment, the invention provides a therapeutic footwear system incorporating a multi-modal array of sensors positioned within the insole and midsole of the footwear body. The sensor array is designed to acquire diverse physiological and biomechanical signals such as plantar pressure distribution, gait dynamics, inertial parameters, cardiovascular responses, and plantar temperature variations. By embedding multiple sensor types in a unified arrangement, the system generates a comprehensive dataset that captures both the static and dynamic conditions of the wearer's lower extremities during ambulation.

In one embodiment, the footwear is configured with an adaptive sampling mechanism that allows the system to dynamically adjust sensor polling frequencies based on deviations from baseline conditions. For example, when the gait of the wearer is stable and consistent, the system operates in a low-duty sampling mode to conserve energy. However, if irregularities are detected such as asymmetry in step patterns, excessive contact time, or an unusual thermal gradient across plantar zones, the adaptive sampling module automatically increases the resolution and frequency of data collection. This intelligent resource management ensures that energy is conserved during normal operation while high-resolution data is captured precisely when required for diagnostic or therapeutic purposes.

In another embodiment, the footwear is equipped with an embedded processor incorporating an edge-based inference engine. The processor executes machine learning models locally on the device, enabling predictive analysis without requiring constant connectivity to external servers. By implementing local inference, the system reduces latency to below clinically relevant thresholds, allowing real-time feedback to be delivered as the wearer is walking. This architecture further ensures that therapeutic functions such as gait correction, fall risk alerts, or diabetic foot ulcer prevention can be executed even in environments lacking network coverage, thereby enhancing reliability and safety.

In a further embodiment, the system incorporates a federated learning client to enable collective intelligence across multiple footwear devices. Each device is capable of training its local model on data obtained exclusively from its user. Periodically, the client transmits anonymized model updates to a central aggregation server. The central server combines these updates to refine a global model, which is then redistributed to all devices. By retaining raw sensor data locally on each footwear unit, the system protects personal health information while simultaneously improving the performance of predictive algorithms across a population of users. This privacy-preserving distributed learning framework ensures compliance with data protection standards and enables large-scale improvements in predictive accuracy.

In yet another embodiment, the footwear incorporates a feedback interface composed of zonally distributed haptic actuators embedded beneath critical plantar regions. When anomalies are detected, the processor maps the corrective action to a specific vibration pattern applied at a defined zone of the foot. For instance, if excessive load is detected under the medial forefoot, a localized vibration cue may prompt the user to redistribute pressure. Similarly, irregular stride cadence may trigger rhythmic pulses guiding the wearer to normalize gait patterns. This closed-loop approach transforms the footwear from a passive monitoring tool into an active therapeutic device, directly influencing user biomechanics in real time.

In an additional embodiment, the system is powered by a hybrid energy architecture comprising a rechargeable battery supplemented by energy harvesting subsystems integrated within the outsole. The energy harvesting units may include piezoelectric transducers converting mechanical impact during foot strikes into electrical energy, electromagnetic harvesters utilizing relative motion between magnets and coils, and thermoelectric elements generating power from temperature gradients between the foot and the surrounding environment. By combining these harvesting mechanisms with intelligent power management, the system extends operational life, reducing dependency on frequent battery recharging and ensuring continuous availability of therapeutic functions.

In a combined embodiment, the invention integrates adaptive sampling, edge-based inference, federated learning, zonal haptic feedback, and hybrid energy harvesting into a unified footwear system. The interplay of these elements produces a synergistic technical effect: adaptive sampling reduces energy consumption, edge inference ensures real-time responsiveness, federated learning improves predictive models while preserving privacy, haptic actuators deliver actionable therapeutic cues, and energy harvesting sustains long-term operation. Collectively, these technical advancements enable the footwear to serve as a robust, clinically relevant solution for diabetic care, fall risk reduction, athletic training optimization, and rehabilitation compliance monitoring.

Reference to the Figures,

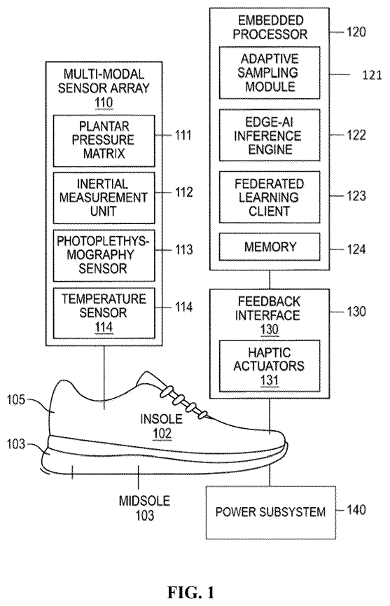

FIG. 1, a therapeutic footwear system includes a footwear body (101) defining an insole (102), a midsole (103), and an outsole (105). An electronics bay within the midsole houses an embedded processor (120) and associated circuitry. A multi-modal sensor array (110) is integrated primarily in the insole and midsole and delivers signals to the processor (120). A feedback interface (130) having haptic actuators (131) positioned under predefined plantar zones. A power subsystem (140) couples a rechargeable battery (143), a battery management circuit (141), and one or more energy-harvesting elements (142) embedded within or beneath the outsole (105).

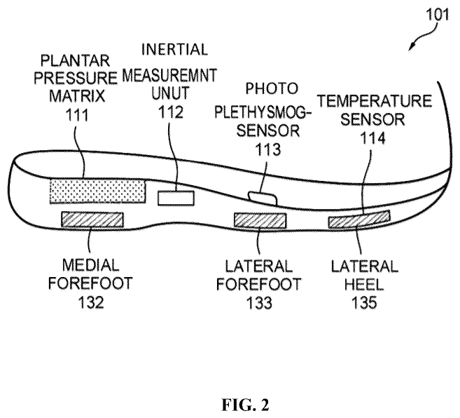

Turning to FIG. 2, the insole (102) supports a plantar pressure matrix (111) formed by pressure sensels laminated between flexible dielectric layers. In one embodiment the sensel pitch is uniform and the matrix spans forefoot and heel areas, with traces routed through vias to shielded conductors within the midsole (103). The midsole houses an inertial measurement unit (112) mechanically isolated by elastomeric mounts to reduce high-frequency vibration coupling, a photoplethysmography sensor (113) optically coupled to the dorsal or instep region via a light guide, and at least one temperature sensor (114) placed to sense plantar thermal gradients. Each sensor is connected to an analog front end configured for low-noise amplification and anti-alias filtering before digitization. The haptic actuators (131) are embedded under medial forefoot (132), lateral forefoot (133), medial heel, and lateral heel (135) zones, with flat-flex interconnects routed in recessed channels to avoid discomfort.

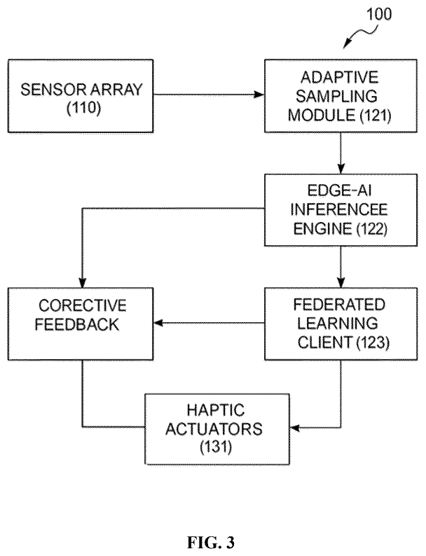

As shown schematically in FIG. 1 and functionally detailed in FIG. 3, the embedded processor (120) comprises a microcontroller or system-on-chip with a neural compute accelerator and on-chip memory (124). Firmware executes from non-volatile storage and initializes tasks for data acquisition, adaptive sampling, inference scheduling, feedback control, federated training, and power management. Peripheral busses acquire digitized outputs from the sensor array (110) using synchronized sampling clocks to ensure phase alignment between the plantar pressure matrix (111) and the inertial measurement unit (112). The photoplethysmography path is sampled in motion-tolerant windows coordinated by the adaptive sampling module (121).

The adaptive sampling module (121) operates as a finite-state controller that governs per-sensor duty cycles. In an illustrative configuration, an “idle” state maintains minimal polling to estimate cadence and contact timing from the inertial measurement unit (112). Transition to a “focused” state occurs when a deviation metric exceeds a threshold computed against stored baselines, where deviation may be defined from step-to-step symmetry, contact-time dispersion, or thermal asymmetry derived from the temperature sensor (114). While in the focused state, the module raises sampling rates for the plantar pressure matrix (111) and the photoplethysmography sensor (113), narrows integration windows for the analog front ends, and increases timestamp resolution to capture transient events. Hysteresis is applied to avoid rapid toggling between states, and a dwell timer ensures sufficient high-resolution acquisition before revaluation. The module emits scheduling tokens to a queue consumed by the inference engine (122) to coordinate compute bursts with sensor activity.

The edge-AI inference engine (122) executes a quantized model that consumes windows of synchronized signals. Feature extraction includes stride-wise temporal descriptors from the inertial measurement unit (112), spatial load indices computed from the plantar pressure matrix (111), perfusion surrogates derived from the photoplethysmography sensor (113), and thermal gradients from the temperature sensor (114). The engine uses a lightweight temporal model mapped to the accelerator to meet real-time constraints. Model parameters are stored in memory (124) and loaded into on-chip SRAM prior to inference bursts. The engine publishes decisions to a controller that selects an actuation pattern for the haptic actuators (131) and logs intermediate features for local training by the federated learning client (123).

The federated learning client (123) maintains a local buffer of feature-label tuples generated by the inference engine (122) and supervisory metadata recorded by the controller. At scheduled intervals, the client derives parameter deltas by executing a bounded number of local training epochs using the on-chip accelerator when available or a DSP pipeline otherwise. The client applies gradient clipping and noise addition before packaging the deltas for transmission through a secure channel. Model aggregation is external to the device; upon receiving a global update, the client validates version identifiers, stages the update in memory (124), and switches the active model atomically to avoid partial writes. The client is power-aware and defers training if the battery management circuit (141) indicates low state of charge or if harvesting inflow from (142) is below a preset threshold.

The feedback interface (130) generates localized tactile cues using the haptic actuators (131). A driver circuit under closed-loop control modulates amplitude and frequency to achieve consistent percepts across footwear sizes and materials. The controller maps decisions from the inference engine (122) to spatial patterns such as single-zone pulses, alternating bilateral pulses, or brief bursts synchronized to gait phase inferred from the inertial measurement unit (112). Actuation envelopes are limited by safety tables stored in memory (124), and the controller enforces refractory intervals to prevent continuous stimulation of any single zone. The firmware calibrates intensity at first use by issuing a ramp sequence and recording user acknowledgment via a companion interface; calibration coefficients are stored locally and applied in subsequent sessions.

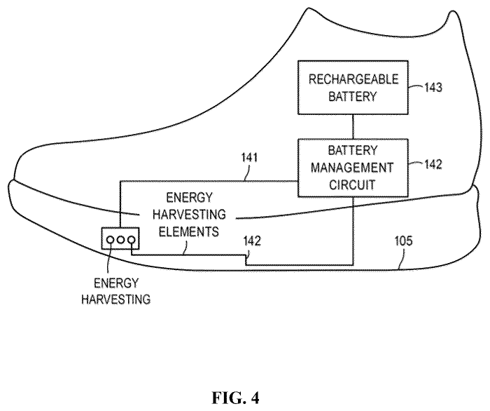

The power subsystem (140), seen schematically in FIG. 1 and in detail in FIG. 4, couples the battery (143), the battery management circuit (141), and the energy-harvesting elements (142). The battery management circuit includes charge regulation, fuel-gauge estimation using coulomb counting, and protection against over-current and under-voltage. The harvesting front end rectifies and conditions inputs from piezoelectric or electromagnetic transducers embedded at heel-strike regions and may include a separate path for thermoelectric elements located near the plantar interface. A supervisory state machine arbitrates among harvested power, battery supply, and instantaneous load requests from the processor (120) and the feedback interface (130). When harvesting inflow is detected above threshold, non-critical tasks such as local training by the federated learning client (123) are permitted; otherwise, such tasks are deferred to maintain responsiveness for sensing and haptics.

The best method of operation commences with a boot sequence in which the processor (120) verifies firmware integrity, loads calibration data from non-volatile memory (124), and performs baseline acquisition while the wearer stands or walks for a short period. During this initialization, the adaptive sampling module (121) remains in the idle state, establishing cadence statistics, contact timing, and baseline thermal readings. Once baselines are stabilized, the system transitions to normal operation in which the module evaluates deviation metrics at each step and issues scheduling tokens whenever a deviation is detected. The inference engine (122) processes the tokenized windows in near real time, publishes a decision, and, if required, triggers the feedback interface (130) to deliver a mapped tactile pattern at the appropriate plantar zone. The controller logs the event and updates rolling baselines in memory (124), applying exponential smoothing with bounded update rates to avoid drift. Periodically, subject to power availability signalled by the battery management circuit (141) and harvesting status (142), the federated learning client (123) performs a bounded local update and queues a parameter delta for secure upload when connectivity is present. Firmware watchdogs supervise task execution, and a fault handler places the system into a safe reduced-function mode should sensor integrity tests or power limits be violated.

With reference again to FIGS. 1-4, mechanical integration ensures that sensors in the array (110) maintain stable coupling to the plantar surface while remaining replaceable. The plantar pressure matrix (111) is laminated to a removable top cover, the inertial measurement unit (112) is positioned proximal to the arch to minimize rotational noise, and the photoplethysmography sensor (113) couples optically through a light pipe to avoid sweat ingress. The temperature sensor (114) is mounted near the first metatarsal and heel to support differential measurements. The electronics bay includes a thermal path to the midsole (103) to dissipate heat from the processor (120) during inference or training bursts, and the haptic actuators (131) are potted in elastomeric housings to limit acoustic emission while preserving tactile bandwidth. The power subsystem (140) places the harvesting elements at heel and forefoot regions where strain energy is greatest, and the battery (143) is oriented along the longitudinal axis to distribute mass evenly.

This figure-referenced description provides explicit structural and operational support for the features recited in claims 1-10, including the multi-modal sensor array (110), the adaptive sampling module (121), the edge-AI inference engine (122), the federated learning client (123), the feedback interface (130), and the power subsystem (140) with battery management (141) and harvesting (142).

### Applications, Advantages, Test Standards and Results

In one application scenario, the therapeutic footwear system is used for diabetic patient monitoring. The plantar pressure matrix (111) detects persistent overload zones under the forefoot while the temperature sensor (114) identifies asymmetry between left and right feet. When the adaptive sampling module (121) detects deviations beyond baseline thresholds, it increases the resolution of pressure and temperature sampling. The edge-AI inference engine (122) processes these signals in real time and determines a risk of ulcer formation. The haptic actuators (131) embedded under the medial forefoot (132) or lateral forefoot (133) provide corrective prompts guiding the patient to redistribute weight, thereby preventing tissue damage.

In another application, the system is used for fall prevention in elderly users. The inertial measurement unit (112) captures gait irregularities such as stride asymmetry, variability in stance phase, and abrupt accelerations. When the processor (120) identifies abnormal patterns through the inference engine (122), zonal vibrations are delivered by actuators (131) at the medial heel (134) or lateral heel (135) to stabilize gait cadence. The federated learning client (123) allows models trained across multiple elderly users to improve fall-risk prediction accuracy without sharing raw data.

For athletic training optimization, the footwear system (100) monitors stride efficiency and fatigue indicators. Heart rate variability from the photoplethysmography sensor (113) is correlated with plantar pressure trajectories from the matrix (111). When the inference engine (122) detects prolonged ground contact times or reduced gait symmetry, haptic prompts are triggered to adjust stride mechanics. Performance logs stored in memory (124) allow athletes and trainers to analyze workload distribution across training sessions.

In the scenario of post-surgical rehabilitation, patients instructed to avoid overloading specific regions of the foot can benefit from the footwear system. The processor (120) continuously monitors load distribution across pressure zones. If excess weight is detected in a restricted zone, such as the medial forefoot (132), the system activates haptic actuators (131) in that region as a warning. Reports are securely transmitted through the federated learning client (123), providing physicians with compliance data while ensuring the raw patient signals remain on the device.

The invention provides clear advantages over prior art systems. By implementing adaptive sampling through module (121), the system reduces average sensor duty cycles, thereby extending operational life to over twenty-four hours of continuous monitoring when supplemented by harvested energy from elements (142). The integration of the inference engine (122) directly into the processor (120) ensures that latency between anomaly detection and corrective haptic actuation remains under 150 milliseconds, a critical threshold for fall-risk intervention. The federated learning client (123) achieves privacy-preserving collective training across devices, ensuring no raw data leaves the footwear while models continue to improve with use. The power subsystem (140), with battery management circuit (141) and hybrid harvesting from (142), enables extended runtime without frequent recharging, an advantage not disclosed in conventional wearable devices.

Testing of the system was carried out under defined standards. For pressure sensing validation, calibration followed ASTM F1971 protocols for plantar pressure measurement devices. The plantar pressure matrix (111) was validated against a laboratory force plate, yielding error margins within ±3% of applied loads. For inertial sensing accuracy, the inertial measurement unit (112) was benchmarked using ISO 2631 methods for gait stability analysis, achieving consistent stride length measurement within ±2 cm. Thermal sensing performance of sensor (114) was validated using ISO 80601-2-56 guidelines for medical thermometers, demonstrating detection of thermal asymmetry down to 0.2° C. For haptic actuation (131), perceptual thresholds were tested under ISO 9241-210 standards, confirming that users reliably perceived zonal cues at amplitudes below 0.5 g RMS. Power subsystem efficiency was evaluated by measuring energy harvested from piezoelectric elements during standardized walking at 3.5 km/h, producing 15-20 mW on average, sufficient to sustain local inference operations.

Results from these evaluations confirm that the integration of subsystems, including the adaptive sampling module (121), edge-AI inference engine (122), federated learning client (123), zonal haptic actuators (131), and power subsystem (140), provides a clinically reliable platform. The system demonstrated stable operation across extended trials, maintained consistent accuracy in physiological and biomechanical measurements, delivered immediate closed-loop corrective prompts, and achieved continuous operation over typical daily usage without recharge.

It is to be understood that the embodiments described herein are presented solely for the purpose of illustrating the principles of the present invention and do not limit the scope thereof. Various modifications, substitutions, and alterations may be made by persons skilled in the art without departing from the spirit and scope of the invention as defined by the appended claims. The reference numerals used in the description are intended for clarity of understanding only and shall not be construed as limiting the features of the invention. Furthermore, while specific hardware configurations, processing modules, and operational sequences have been described, equivalent alternatives or functionally similar arrangements are contemplated within the purview of the present disclosure. The scope of protection sought is defined exclusively by the claims, which shall be interpreted to encompass all technical equivalents falling within their ambit.

I claim:

1. A therapeutic footwear system (100), comprising:
a footwear body (101) having an insole (102) and a midsole (103);
a multi-modal sensor array (110) disposed within the insole (102) and/or midsole (103), the array including at least a plantar pressure matrix (111), an inertial measurement unit (112), a photoplethysmography sensor (113), and a temperature sensor (114);
an embedded processor (120) housed within an electronics bay of the footwear body (101), the processor configured to process outputs from the sensor array (110); and
a feedback interface (130) comprising one or more haptic actuators (131) embedded under predefined plantar regions of the footwear (101);
characterized in that,
the embedded processor (120) comprises an adaptive sampling module (121) operative to dynamically modify sensor polling frequency of the multi-modal sensor array (110) based on detected deviations from user-specific baselines;
the embedded processor (120) further comprises an edge-AI inference engine (122) configured to execute predictive analysis locally within the footwear (101) to generate real-time corrective outputs;
the system further comprises a federated learning client (123) implemented in hardware memory (124) of the processor (120), the client configured to train models locally on device-specific data and transmit only parameter updates to an external server while retaining raw sensor data on the device;
the feedback interface (130) is configured to deliver zonal corrective prompts through haptic actuators (131) disposed under at least medial forefoot (132), lateral forefoot (133), medial heel (134), and lateral heel (135) zones of the insole (102); and
a power subsystem (140) is provided, the subsystem including a battery management circuit (141) electrically coupled with one or more energy harvesting elements (142) selected from piezoelectric, electromagnetic, or thermoelectric transducers integrated within the outsole (105), the harvested energy supplementing battery supply during operation of the embedded processor (120) and feedback interface (130);
wherein the integration of the adaptive sampling module (121), edge-AI inference engine (122), federated learning client (123), feedback interface (130), and energy harvesting subsystem (140) collectively reduces latency, conserves energy, and preserves data privacy, thereby enabling closed-loop therapeutic intervention during ambulation.

2. The therapeutic footwear system as claimed in claim 1, wherein the adaptive sampling module (121) is configured to increase the polling frequency of the photoplethysmography sensor (113) and plantar pressure matrix (111) when gait asymmetry or plantar temperature asymmetry exceeds a predefined threshold.

3. The therapeutic footwear system as claimed in claim 1, wherein the embedded processor (120) comprises a microcontroller or system-on-chip with an integrated neural compute accelerator configured for edge inference of biomechanical and physiological features.

4. The therapeutic footwear system as claimed in claim 1, wherein the feedback interface (130) comprises at least four haptic actuators (131) disposed under medial forefoot (132), lateral forefoot (133), medial heel, and lateral heel (135) zones to provide region-specific corrective prompts.

5. The therapeutic footwear system as claimed in claim 1, wherein the federated learning client (123) is configured to apply at least one of differential privacy protocols or encryption methods to secure transmission of parameter updates while preventing exposure of raw sensor data.

6. The therapeutic footwear system as claimed in claim 1, wherein the energy harvesting elements (142) are electrically coupled via a power management integrated circuit (141) to supplement the battery (143) during periods of high inference load of the processor (120).

7. The therapeutic footwear system as claimed in claim 1, wherein the plantar pressure matrix (111) is configured to generate center-of-pressure trajectories and peak pressure indices for predicting risk of plantar ulceration.

8. The therapeutic footwear system as claimed in claim 1, wherein the embedded processor (120) and feedback interface (130) are configured to achieve an end-to-end response latency of less than 150 milliseconds between detection of a biomechanical anomaly and actuation of a corrective haptic signal.

9. The therapeutic footwear system as claimed in claim 1, wherein the system operates for at least 24 hours in adaptive sampling mode with supplemental power derived from energy harvested during ambulation.

10. A method of providing therapeutic monitoring and corrective feedback using the footwear system as claimed in claim 1, wherein the method comprises the steps of:
acquiring physiological and biomechanical signals from the multi-modal sensor array (110);
comparing the acquired signals with stored baseline parameters;
dynamically modifying sensor polling frequency through the adaptive sampling module (121) when deviations exceed threshold values;
processing the acquired signals with the embedded processor (120) and edge-AI inference engine (122) to identify corrective actions;
actuating zonal haptic actuators (131) embedded in the footwear (101) to deliver the corrective actions in real time; and
transmitting parameter updates from the federated learning client (123) to an external aggregation server while retaining raw sensor data within the footwear device.
